SEQUENCE LISTING (10 sequences)

SEQ ID NO: 1 - protein, 21 aa, Escherichia coli
PQRCPSLRQAVQLTHQQQRQV

SEQ ID NO: 2 - protein, 31 aa, Escherichia coli
RCGQQLRNISPPQRCPSLRQAVQLTHQQQGQ

SEQ ID NO: 3 - protein, 21 aa, Escherichia coli
PQRCPSLRQAVQLTHQQQGQV

SEQ ID NO: 4 - protein, 16 aa, Escherichia coli
PQRCPSLRQAVQLTHQ

SEQ ID NO: 5 - protein, 22 aa, Escherichia coli
QGPGRQPDFQRCGQQLRNISPP

SEQ ID NO: 6 - protein, 60 aa, Escherichia coli
QGPGRQPDFQRCGQQLRNISPPQRCPSLRQAVQLTHQQQGQVGPQQVRQMYRVASNIPST

SEQ ID NO: 7 - protein, 21 aa, Escherichia coli
GQVGPQQVRQMYRVASNIPST

SEQ ID NO: 8 - protein, 11 aa, Escherichia coli
PQRCPSLRQAV

SEQ ID NO: 9 - protein, 11 aa, Escherichia coli
SLRQAVQLTHQ

SEQ ID NO: 10 - protein, 12 aa, Escherichia coli
AVQLTHQQQGQV

(19) United States
(12) Patent Application Publication
(10) Pub. No.: US 2007/0099838 A1
(43) Pub. Date: May 3, 2007

(54) Plant-derived peptides harboring water-cleaning and antimicrobial activities

(75) Inventors: Nicolas Mermod, Buchillon (CH); Mougli Suarez, Ecublens (CH)

Correspondence Address: NIXON & VANDERHYE, PC, 901 NORTH GLEBE ROAD, 11TH FLOOR, ARLINGTON, VA, 22203, US

(73) Assignee: OPTIMA ENVIRONNEMENT S.A., Nyon (CH)

(21) Appl. No.: 10/568,827

(22) PCT Filed: Aug. 23, 2004
(86) PCT No.: PCT/CH04/00536

§ 371 Date: Aug. 18, 2006

(30) Foreign Application Priority Data
Aug. 22, 2003 (WO) PCT/CH03/00568

(51) Int. Cl. A61K 38/10; C07K 14/47

(52) U.S. Cl. 514/13; 530/326

(57) ABSTRACT

Protein family derived from the protein defined by the amino acid sequence of QGPGRQPDFQRCGQQLRNISPPQRCPSLRQAVQLTHQQQGQVGPQQVRQMYRVAS NIPST (SEQ ID NO:6) is described.

The present application claims a priority based on international patent application PCT/03/00568. The content of said priority application is hereby incorporated by reference.

### FIELD OF THE INVENTION

The present invention relates to a family of proteins which may be used for different purposes such as coagulation agents for water treatment or as antimicrobial agents.

### STATE OF THE ART

A protein corresponding to the above-cited definition, called FLO, is disclosed in PCT patent application WO 03/008441 A2 (OPTIMA ENVIRONMENT S.A.).

### SUMMARY OF THE INVENTION

The present invention concerns derivatives of FLO which, surprisingly, show similar or higher coagulating or antimicrobial activities than FLO.

The inventors have also unexpectedly found that some of those derivatives may show either a coagulating or an antimicrobial activity.

Finally, it was found that other FLO derivatives had neither a coagulating nor an antimicrobial activity.

### DETAILED DESCRIPTION OF THE INVENTION

Table 1 summarizes the derivatives of FLO representing the object of the invention.

The following terms are used:

+++higher activity than FLO

++equivalent activity of FLO

+lower activity than FLO

−no activity observed

More information regarding the detailed description of the invention (e.g. material & methods, experimental results) can be found in international patent application PCT/CH03/00568 which is incorporated by reference.

TABLE 1

| | |
|---|---|
| Coag. | antlbt. |

| | | |
|---|---|---|
| Flo | | |
| QGPGRQPDFQRCGQQLRNISPPQRCPSLRQAVQLTH | ++ | ++ |
| QQQGQVGPQQRQMYRVASNIPST | | |

```
predicted alpha helices
XXXXXXXXXXXXXX XXXXXXXXXXXXXXXXX XXXXXXXXXXXXXX
Positive charge
    + + + + + (+) + +
Glutamine (Gln)
Q Q Q QQ Q Q Q QQQ QQ Q
Hydrophobic
          F C L I C L AV L V V M VA I
Gln/Hydrophobic
Q Q FQ C QQL I Q C L QAVQL QQQ QV QQV QM
VA I
```

| | | |
|---|---|---|
| P1 | | |
| QGPGRQPDFQRCGQQLRNISPP | − | +/− |
| P2 | | |
| PQRCPSLRQAVQLTHQQQGQV | ++ | + |
| P3 | | |
| GQVGPQQVRQMYRVASNIPST | − | − |
| P2.1 | | |
| RCGQQLRNISPPQRCPSLRQAVQLTHQQQGQ | − | ++++ |
| P2a | | |
| PQRCPSLRQAV | − | − |
| P2b | | |
| SLRQAVQLTHQ | − | − |
| P2c | | |
| AVQLTHQQQGQV | − | − |
| P2ab | | |
| PQRCPSLRQAVQLTHQ | ++ | +/− |
| P2GR40 | | |
| PQRCPSLRQAVQLTHQQQRQV | +++ | ++ |

1. A protein (P2GR40) defined by the amino acid sequence of

| | |
|---|---|
| PQRCPSLRQAVQLTHQQQRQV. | (SEQ ID NO:1) |

2. Use of the protein of claim 1 as a coagulating agent.

3. Use of the protein of claim 1 as an antimicrobial agent.

4. A protein (P2.1) defined by the amino acid sequence of

| | |
|---|---|
| RCGQQLRNISPPQRCPSLRQAVQLTHQQQGQ. | (SEQ ID NO:2) |

5. Use of the protein of claim 4 as an antimicrobial agent.

6. A protein (P2) defined by the amino acid sequence of

| | |
|---|---|
| PQRCPSLRQAVQLTHQQQGQV. | (SEQ ID NO:3) |

7. Use of the protein of claim 6 as a coagulating agent.

8. Use of the protein of claim 6 as an antimicrobial agent.

9. A protein (P2ab) defined by the amino acid sequence of

| | |
|---|---|
| PQRCPSLRQAVQLTHQ. | (SEQ ID NO:4) |

10. Use of the protein of claim 9 as a coagulating agent.

11. Use of the protein of claim 9 as a antimicrobial agent.

12. A protein (P1) defined by the amino acid sequence of

| | |
|---|---|
| QGPGRQPDFQRCGQQLRNISPP. | (SEQ ID NO:5) |

13. Use of the protein of claim 12 as an antimicrobial agent.
